(19) United States
(12) Patent Application Publication
(10) Pub. No.: US 2024/0256225 A1
(43) Pub. Date: Aug. 1, 2024

(54) SYSTEMS AND METHODS FOR NORMALIZATION OF MACHINE LEARNING DATASETS

(71) Applicant: PRENOSIS, INC., Chicago, IL (US)

(72) Inventors: Ishan Taneja, Chicago, IL (US); Carlos G. Lopez-Espina, Chicago, IL (US); Bobby Reddy, Jr., Chicago, IL (US); Sihai Dave Zhao, Chicago, IL (US); Ruoqing Zhu, Chicago, IL (US); Akhil BHARGAVA, Chicago, IL (US)

(21) Appl. No.: 18/018,836

(22) PCT Filed: Aug. 6, 2021
(86) PCT No.: PCT/US2021/044943

§ 371 Date: Jan. 30, 2023

Related U.S. Application Data
(60) Provisional application No. 63/062,240, filed on Aug. 6, 2020.

(51) Int. Cl. G06F 7/76; G16H 10/40

(52) U.S. Cl. CPC G06F 7/76; G16H 10/40

(57) ABSTRACT

A system for biomarker data normalization in training datasets. The system includes one or more processors and one or more memory devices storing instructions that configure the one or more processors to perform operations. The operations may include receiving, data files comprising biomarker records (each of the biomarker records comprising a plurality of metadata fields), identifying a template record for normalization, the template record comprising template metadata fields, and generating a normalization vector comprising mismatching biomarker records. The operations may also include identifying adjustment functions for each one of the plurality of metadata fields, modifying data fields of biomarker records in the normalization vector by applying the adjustment functions, and generating a normalized data file comprising the modified biomarker records.

### CROSS-REFERENCE TO RELATED APPLICATIONS

The present application claims priority to and the benefit of the U.S. Provisional Patent Application No. 63/062,240, filed Aug. 6, 2020, titled “Systems and Methods for Normalization of Machine Learning Training Data,” which is hereby incorporated by reference in its entirety as if fully set forth below and for all applicable purposes.

### TECHNICAL FIELD

The present disclosure generally relates to systems and methods for normalizing electronic data and, more particularly, to systems and methods for normalizing health data, such as biomarker records, used for machine learning training.

### BACKGROUND

Normalization is a technique often applied as part of data preparation for machine learning. The goal of normalization is to change the values in a dataset to a common scale or parameter, without distorting differences in the ranges of values. For machine learning, every dataset does not require normalization. However, normalization is required when features have different ranges and/or scales because raw data frequently includes attributes with varying scales. For example, one attribute may be in kilograms and another may be in pounds, or in a count. In many machine learning applications, normalization is important to obtain consistent results. Normalization is a good technique to use especially when you do not know the distribution of your data or when you know the distribution is not Gaussian (a bell curve).

Normalizing data is particularly challenging in applications that employ very large and diverse datasets for training and/or validation. When the normalization depends on multiple factors and/or characteristics, the normalization can be complex and computationally expensive. For instance, normalizing datasets based on multiple factors, history of data, and different weighting, can be complex and computationally expensive. Further, in some scenarios it is not sufficient to use standard normalization for successful modeling and it is necessary to discard certain data points that cannot be properly normalized. This loss of data is highly undesirable, especially in applications in which collecting data is expensive.

The disclosed systems and methods address one or more of the problems set forth above and/or other problems in the prior art.

### SUMMARY

One aspect of the present disclosure is directed to a system for biomarker data normalization in training datasets. The system may include one or more processors and one or more memory devices storing instructions that configure the one or more processors to perform operations. The operations may include receiving a data file comprising a biomarker record that includes a plurality of biomarker metadata fields; identifying a template record for normalizing the biomarker record, the template record comprising template metadata fields; generating a normalization vector comprising a mismatching metadata field that mismatches a corresponding template metadata field in the template record; identifying an adjustment function for the mismatching metadata fields; modifying a data field of the biomarker record by applying the adjustment function to the data field, the data field corresponding to the mismatching metadata field in the normalization vector; and generating a normalized data file comprising the modified biomarker record.

Another aspect of the present disclosure is directed to a computer implemented method for biomarker data normalization in training data sets. The method includes receiving a data file comprising a biomarker record that includes a plurality of biomarker metadata fields; identifying a template record for normalizing the biomarker record, the template record comprising template metadata fields; generating a normalization vector comprising a mismatching metadata field that mismatches a corresponding template metadata field in the template record; identifying an adjustment function for the mismatching metadata fields; modifying a data field of the biomarker record by applying the adjustment function to the data field, the data field corresponding to the mismatching metadata field in the normalization vector; and generating a normalized data file comprising the modified biomarker record.

Yet another aspect of the present disclosure is directed to a non-transitory computer-readable medium storing instructions that when executed by one or more processors, cause the one or more processors to perform operations. The operations may include receiving a data file comprising a biomarker record that includes a plurality of biomarker metadata fields; identifying a template record for normalizing the biomarker record, the template record comprising template metadata fields; generating a normalization vector comprising a mismatching metadata field that mismatches a corresponding template metadata field in the template record; identifying an adjustment function for the mismatching metadata fields; modifying a data field of the biomarker record by applying the adjustment function to the data field, the data field corresponding to the mismatching metadata field in the normalization vector; and generating a normalized data file comprising the modified biomarker record.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an exemplary architecture suitable for implementing machine learning methods, in accordance with disclosed embodiments.

FIG. 2 illustrates a block diagram of an exemplary server and client, according to disclosed embodiments.

FIG. 3 illustrates a block diagram of an exemplary machine learning system, in accordance with disclosed embodiments.

FIG. 4 illustrates a block diagram of an exemplary system for normalizing data files, in accordance with various embodiments.

FIG. 5 illustrates a block diagram of an exemplary record file with patient data, in accordance with disclosed embodiments.

FIG. 6 illustrates a flow chart of a method for normalizing biomarker records, in accordance with disclosed embodiments.

FIG. 7 illustrates a flow chart of a method for identifying unnormalized records based on metadata information, in accordance with disclosed embodiments.

FIG. 8 illustrates a flow chart of a method for filtering records based on metadata information, in accordance with disclosed embodiments.

FIG. 9 illustrates a flow chart of a method for deployment of normalization, in accordance with disclosed embodiments.

FIG. 10 illustrates a flow chart of a method for applying normalization functions, in accordance with disclosed embodiments.

FIG. 11 illustrates an exemplary adjustment function table, in accordance with disclosed embodiments.

FIG. 12 illustrates a flow chart of a method for calculating errors based on missing metadata fields, in accordance with disclosed embodiments.

FIG. 13 illustrates a timeline of blood draw records, in accordance with disclosed embodiments.

FIG. 14 is a block diagram illustrating an example computer system with which the client and server of FIGS. 1 and 2, and the methods of FIGS. 6-10 can be implemented, in accordance with various embodiments.

FIG. 15 illustrates an example neural network that can be used to implement a logic engine, in accordance with various embodiments.

In the figures, elements and steps denoted by the same or similar reference numerals are associated with the same or similar elements and steps, unless indicated otherwise.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

Machine learning (ML) models often face the challenge of normalizing data for either training or validation, especially when the normalization requires the consideration of multiple parameters and must be performed quickly (e.g., in real-time environments). Traditional ML, artificial intelligence (AI), and neural network (NN) algorithms are trained using a large amount of data inputs prior to analysis. Accordingly, systems using any of the above algorithms desirably have complete sets of input data available before evaluation using the trained ML/AI/NN algorithms. However, in a streaming data environment or other time-sensitive configurations, data may need to be normalized quickly and efficiently to be able to use the data during training or validation tasks.

The problem of performing data normalization in time-sensitive analysis and/or streaming data environment involves optimizing processes and normalization functions. This problem is exacerbated when normalization parameters are dynamic because normalization with dynamic templates or parameters may require specialized normalization methods and evaluation of multiple parameters concurrently. Such complexity, results in a computational problem that requires specific methods to minimize computer resources during data normalization. In various embodiments, a solution to this problem includes methods and systems to normalize data and feeding such normalized data to ML systems.

Embodiments as disclosed herein provide a solution to the above problem in the form of automated systems and methods for data normalization. In various embodiments, a normalization engine or system identifies data characteristics based on metadata associated with the data to identify one or more normalization functions and how these normalization functions should get executed.

Disclosed embodiments may improve the technical field of healthcare data processing by providing tools and methods for efficient normalization of data. In various embodiments, the normalization system may be based on a network that collects, converts, and consolidates data from health centers into a normalized format that can be used as training datasets. For example, various embodiments may enable the normalization of biomarker records. Biomarker records may include results of measurements performed on biomarkers. In various embodiments, the term biomarker may refer to a measurable substance and/or characteristic in an organism the presence and/or the measured value of which may be indicative of phenomena such as but not limited to diseases, infections, environmental exposures, tissue/organ function levels, and/or the like. For example, in some instances, a biomarker record may include results of vital measurements, which are measurements performed to measure or detect measurable substances and/or characteristics of an organism the presence and/or the measured value of which can be indicative of the organism's most basic body functions. Examples of said vital measurements include measurements for the vital signs (e.g., body temperature, body temperature, pulse rate, blood pressure, respiration rate, etc.) of an organism.

As another example, in some instances, a biomarker record may include results of laboratory measurements, which are measurements performed on measurable biological atoms, ions, molecules, etc., of an organism the presence and/or the measured value of which can be indicative of a phenomenon that is found in the body of the organism. As noted above, the phenomenon can be a disease, an infection, an environmental exposure, tissue/organ function level, etc. In some instances, laboratory measurements may be measurements performed on samples taken from the organism, examples of which include bodily fluids and/or waste (e.g., blood, urine, feces, etc.). Examples of said laboratory measurements include measurements for white blood cell count, C-reactive proteins (CPR) tests (e.g., indicative of inflammation in the body of the organism), creatinine tests (e.g., indicative of the functioning of kidneys), polymerase chain reaction (PCR) tests (e.g., indicative of SARS-CoV-2 disease), and/or the like.

As yet another example, in some instances, a biomarker record may include results of physical measurements, which are measurements of the physical characteristics of the body of the organism the presence and/or the measured value of which can be indicative of any of the afore-mentioned phenomena. Examples of said physical measurements include measurements for height, eye color, nose width, etc., which can be indicative of phenomena such as but not limited to environments exposure, etc. It is to be understood that the above discussion related to biomarker records is for non-limiting illustration purposes, and that a biomarker record of an organism can include results of any type of measurements performed on any biomarkers of the organism.

Biomarker measurements can be highly complex and dependent on a plurality of parameters which are both dynamic and static. These biomarker measurements are also frequently expensive to collect and highly valuable. Various embodiments of the present disclosure allow for automated normalization of such biomarker information to incorporate it in training or validation datasets for machine learning processes.

Reference will now be made to the accompanying drawings, which describe exemplary embodiments of the present disclosure.

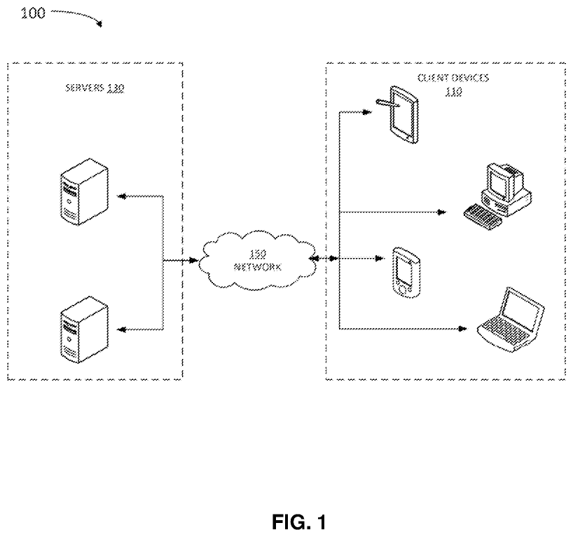

FIG. 1 illustrates an example architecture 100 for a time-sensitive trigger in a streaming data environment, in accordance with various embodiments. Architecture 100 includes servers 130 and client devices 110 connected over a network 150. One of the many servers 130 is configured to host a memory including instructions which, when executed by a processor, cause the server 130 to perform at least some of the steps in methods as disclosed herein. At least one of servers 130 may include, or have access to, a database including clinical data for multiple patients.

Servers 130 may include any device having an appropriate processor, memory, and communications capability for hosting the collection of images and a trigger logic engine. The trigger logic engine may be accessible by various client devices 110 over network 150. Client devices 110 can be, for example, desktop computers, mobile computers, tablet computers (e.g., including e-book readers), mobile devices (e.g., a smartphone or PDA), or any other devices having appropriate processor, memory, and communications capabilities for accessing the trigger logic engine on one of servers 130. In accordance to various embodiments, client devices 110 may be used by healthcare personnel such as physicians, nurses or paramedics, accessing the trigger logic engine on one of servers 130 in a real-time emergency situation (e.g., in a hospital, clinic, ambulance, or any other public or residential environment). In various embodiments, one or more users of client devices 110 (e.g., nurses, paramedics, physicians, and other healthcare personnel) may provide clinical data to the trigger logic engine in one or more server 130, via network 150.

In yet other embodiments, one or more client devices 110 may provide the clinical data to server 130 automatically. For example, in various embodiments, client device 110 may be a blood testing unit in a clinic, configured to provide patient results to server 130 automatically, through a network connection. Network 150 can include, for example, any one or more of a local area network (LAN), a wide area network (WAN), the Internet, and the like. Further, network 150 can include, but is not limited to, any one or more of the following network topologies, including a bus network, a star network, a ring network, a mesh network, a star-bus network, tree or hierarchical network, and the like.

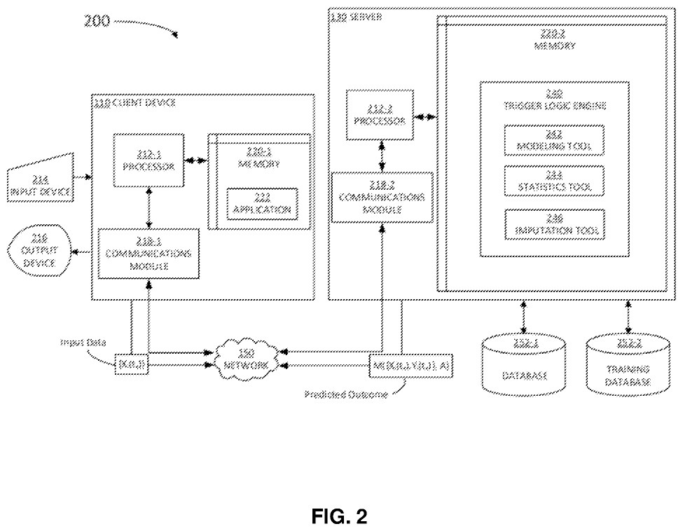

FIG. 2 is a block diagram 200 illustrating an example server 130 and client device 110 in the architecture 100 of FIG. 1, according to certain aspects of the disclosure. Client device 110 and server 130 are communicatively coupled over network 150 via respective communications modules 218-1 and 218-2 (hereinafter, collectively referred to as “communications modules 218”). Communications modules 218 are configured to interface with network 150 to send and receive information, such as data, requests, responses, and commands to other devices on the network. Communications modules 218 can be, for example, modems or Ethernet cards. Client device 110 and server 130 may include a memory 220-1 and 220-2 (hereinafter, collectively referred to as “memories 220”), and a processor 212-1 and 212-2 (hereinafter, collectively referred to as “processors 212”), respectively. Memories 220 may store instructions which, when executed by processors 212, cause either one of client device 110 or server 130 to perform one or more steps in methods as disclosed herein. Accordingly, processors 212 may be configured to execute instructions, such as instructions physically coded into processors 212, instructions received from software in memories 220, or a combination of both.

In accordance with various embodiments, server 130 may include, or be communicatively coupled to, a database 252-1 and a training database 252-2 (hereinafter, collectively referred to as “databases 252”). In one or more implementations, databases 252 may store clinical data for multiple patients. In accordance with various embodiments, training database 252-2 may be the same as database 252-1, or may be included therein. The clinical data in databases 252 may include metrology information such as non-identifying patient characteristics; vital signs; blood measurements such as complete blood count (CBC), comprehensive metabolic panel (CMP), and blood gas (e.g., Oxygen, CO2, and the like); immunologic information; biomarkers; culture; and the like. The non-identifying patient characteristics may include age, gender, and general medical history, such as a chronic condition (e.g., diabetes, allergies, and the like). In various embodiments, the clinical data may also include actions taken by healthcare personnel in response to metrology information, such as therapeutic measures, medication administration events, dosages, and the like. In various embodiments, the clinical data may also include events and outcomes occurring in the patient's history (e.g., sepsis, stroke, cardiac arrest, shock, and the like). Although databases 252 are illustrated as separated from server 130, in certain aspects, databases 252 and trigger logic engine 240 can be hosted in the same server 130, and be accessible by any other server or client device in network 150.

Memory 220-2 in server 130 may include a trigger logic engine 240 for evaluating a streaming data input and triggering an action based on a predicted outcome thereof. Trigger logic engine 240 may include a modeling tool 242, a statistics tool 244, and an imputation tool 246. Modeling tool 242 may include instructions and commands to collect relevant clinical data and evaluate a probable outcome. Modeling tool 242 may include commands and instructions from a neural network (NN), such as a deep neural network (DNN), a convolutional neural network (CNN), and the like. According to various embodiments, modeling tool 242 may include a machine learning algorithm, an artificial intelligence algorithm, or any combination thereof.

Statistics tool 244 evaluates prior data collected by trigger logic engine 240, stored in databases 252, or provided by modeling tool 242. Imputation tool 246 may provide modeling tool 242 with data inputs otherwise missing from a metrology information collected by trigger logic engine 240.

Client device 110 may access trigger logic engine 240 through an application 222 or a web browser installed in client device 110. Processor 212-1 may control the execution of application 222 in client device 110. In accordance with various embodiments, application 222 may include a user interface displayed for the user in an output device 216 of client device 110 (e.g., a graphical user interface—GUI—). A user of client device 110 may use an input device 214 to enter input data as metrology information or to submit a query to trigger logic engine 240 via the user interface of application 222. In accordance with various embodiments, an input data, {Xi(tx)}, may be a 1×n vector where Xij indicates, for a given patient, i, a data entry j (0≤j≤n), indicative of any one of multiple clinical data values (or stock prices) that may or may not be available, and tx indicates a time when the data entry was collected. Client device 110 may receive, in response to input data {Xi(tx)}, a predicted outcome, M({Xi(tx), Yi(tx)}), from server 130. In accordance to various embodiments, predicted outcome M({Xi(tx), Yi(tx)}), may be determined based not only on input data, {Xi(tx)}, but also on an imputed data, {Yi(tx)}. Accordingly, imputed data {Yi(tx)} may be provided by imputation tool 246 in response to missing data from the set {Xi(tx)}. Input device 214 may include a stylus, a mouse, a keyboard, a touch screen, a microphone, or any combination thereof. Output device 216 may also include a display, a headset, a speaker, an alarm or a siren, or any combination thereof.

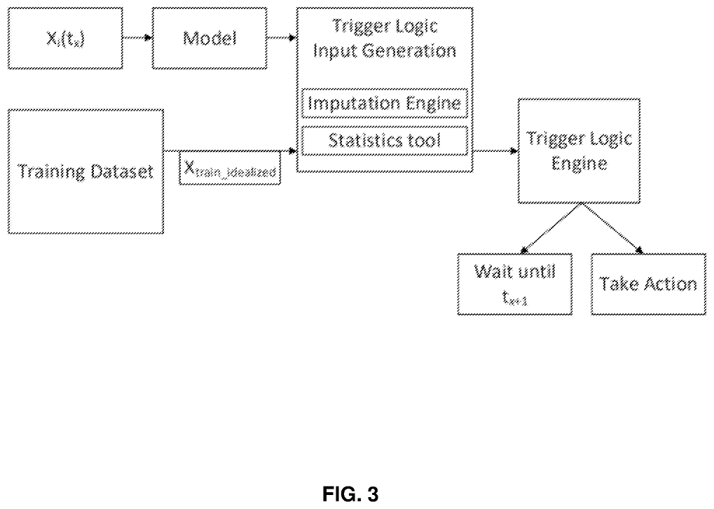

FIG. 3 illustrates a block diagram of a trigger system for a time-sensitive, streaming data environment, in accordance with various embodiments. The trigger system includes a model (hereinafter, designated as M) that provides input data {Xi(tx)} to a trigger logic input generation module. The trigger logic input generation module includes an imputation engine and a statistics tool. The imputation engine provides imputed data {Yi(tx)}. In accordance to various embodiments, the model may include a machine learning model, an artificial intelligence model, a neural network model or any combination thereof, configured to predict an outcome using a training dataset (hereinafter, referred to as Xtrain_idealized). In accordance with various embodiments, Xtrain_idealized is an m by n matrix, where m refers to the number of patients and n refers to the number of features in the clinical data that may be relevant to an outcome for each of the patients. In accordance to various embodiments, for each row in Xtrain_idealized, all features (e.g., clinical data values) may be available (e.g., measured or otherwise provided by medical personnel, a patient, and the like), regardless of the actual time it is available.

In accordance with various embodiments, M is applied to input {Xi(tx)}, wherein the features are assumed to arrive on a streaming basis so, for a given patient i, each feature j arrives at an arbitrary time tx. For each feature, time, tx, may be on a predetermined schedule, asynchronous, or random. The trigger logic engine provides a decision as to whether or not the system should take an action based on metrics (defined later) derived from the statistics tool. In accordance with various embodiments, the trigger logic engine may decide to not take an action at time tx, and then the same process is repeated at time tx+1, when new data Xi(tx+1) may arrive.

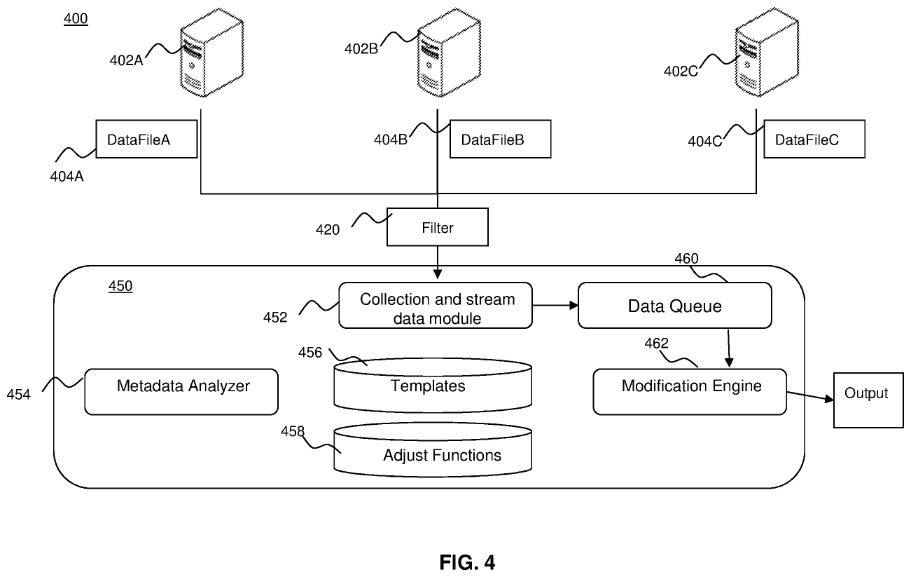

FIG. 4 illustrates a block diagram of an exemplary system 400 for normalizing data files, in accordance with various embodiments. System 400 may include a plurality of data providers 402A, 402B, and 402C. Each of these data providers may be coupled to the normalization system 450. In various embodiments, as shown in FIG. 4, data providers 402A-402C may be connected through a filter 420. In various embodiments, however, there may be no filter 420, or filter 420 may be placed within each one of the individual connections of data providers 402A-402C or within normalization system 450.

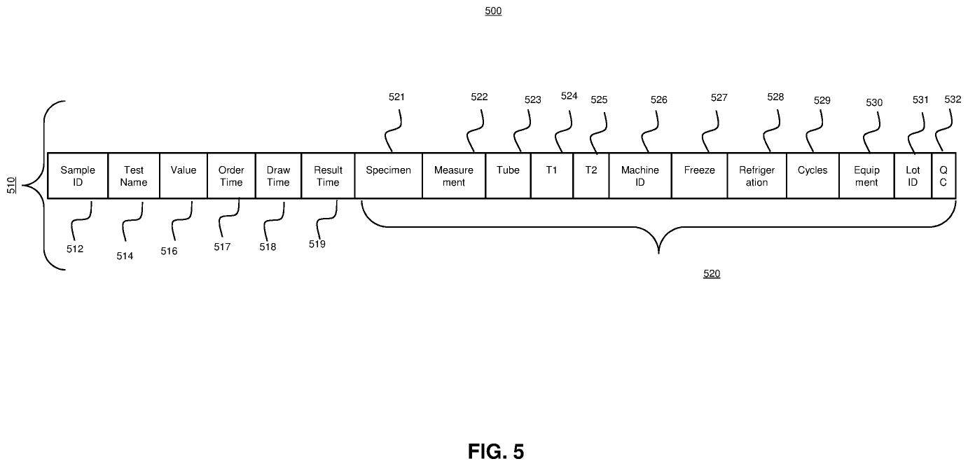

Data providers 402A-402C may send records 404A, 404B, and 404C to normalization system 450. Records 404A-404C may include biomarker records that are associated with metadata. As further discussed in connection with FIG. 5, metadata in records 404A-404C may include multiple metadata fields. The records may be represented through dictionary or array like data structures.

Normalization system 450 may include a collection and stream data module 452, a metadata analyzer 454, a templates memory 456, and an adjust functions memory 458. Further, normalization system 450 may also include a modification engine 462.

Collection and stream data module 452 may include a unified, high-throughput, low-latency platform for handling real-time data feeds. Collection and stream data module 452 may connect to external systems (for data import/export) with a Java, Python, C, or C++ stream processing library. Collection and stream data module 452 may also use a binary TCP-based protocol that is optimized for efficiency and relies on a “message set” abstraction that naturally groups records together. The stream data module can act in a synchronous or asynchronous fashion depending on bandwidth constraints and latency requirements.

The data from the stream data module is then fed into data queue 460, which serves as the ordering scheme (first-in first-out) for processing data. Depending on the specimen, certain sample IDs may be grouped together if they originate from the same test panel and were drawn from the same patient at the same time (e.g. complete blood cell count, complete metabolic panel, etc.).

Metadata analyzer 454 may include hardware or software configured to compare, compile, and/or identify metadata. Metadata analyzer 454 may be implemented with hardware or software components and implement routines for a unified, high-throughput, low-latency platform for handling real-time data feeds. Template memory 456 may include a plurality of template records that include template metadata fields, which may be used by normalization system 450 to identify not normalized records and initiate the normalization process. Template memory 456 may be implemented as a SQL or NoSQL database. If implemented as a SQL database, the columns in the database may correspond to name of the fields in a template record and the test name may correspond to the primary key. If implemented as a NoSQL database, the key may correspond to the test name and the values may correspond to name of the fields in a template record. Adjust functions memory 458 may store normalization functions that, for example, adjust values based on metadata parameters. Such functions may be saved as serialized objects in a Docker container where each function is indexed by its corresponding test name and field name (e.g [IL-6, tube type], [IL-6, machine ID], etc.)

Modification engine 462 may include hardware or software configured to modify records originating from the data queue, adjust functions, template metadata fields, and/or metadata analysis.

FIG. 5 illustrates a block diagram 500 of an exemplary record file with patient data, in accordance with disclosed embodiments. Block diagram 500 shows a record 510 including multiple fields. Record 510 includes a sample ID 512, a test name 514, a value 516, an order time 517, a draw time 518, and a result time 519. In addition, record 510 includes a plurality of metadata fields 520. The plurality of metadata fields 520 may include specimen field 521, a measurement filed 522, a tube field 523, a refrigeration time field 524, a thawing time field 525, a machine ID filed 526, a freeze field 527, a refrigeration field 528, a number of cycles field 528, an equipment field 530, a lot ID field 531, and QC/reincurred sample field 532. In various embodiments, metadata fields 520 may include a type of specimen collected field, the type of specimen collected field indicating at least one of blood, urine, or cerebrospinal fluid. Metadata fields 520 may also include a source of the measurement field (the source measurement field including at least one of vein or artery) along a type of tube field. Moreover, metadata fields 520 may also include a first time lapse field (the time lapse field including a time between sample collection time and measurement time) and a second time lapse field (the second time lapse field including a time between sample measurement time and sample refrigeration). In addition, metadata fields 520 may also include a third time lapse field (the third time lapse field including a time between sample offsite refrigeration and sample offsite freezer placement) and a fourth time lapse (the fourth time lapse including a time between sample onsite freezer time and sample onsite measurement time).

Alternatively, or additionally, metadata fields 520 may also include a machine identifier field and a measurement process field. Further metadata fields 520 may include an offsite refrigeration temperature field, where the offsite refrigeration temperature field including a plurality of temperature values experienced by samples while stored in an offsite refrigerator, and an offsite freezer temperature field, the offsite freezer temperature field including a plurality of temperature values experienced by samples while stored in offsite freezers. Further, metadata fields 520 may include a temperature during transport field, the temperature during transport field including temperature values samples experience while being transported from the offsite freezer to onsite freezer. Further, metadata fields may also include a measurement process field, a number of freeze-thaw cycles fields, and an equipment collection field.

Metadata fields 520 may also include information related to the lot ID that consists of all the unique components (e.g diluents, buffers, antibodies, biologics, recombinant proteins, chemically synthesized substances, etc.) and their associated unique IDs that were used to measure a given sample. Additionally, metadata fields 520 may include the concentration of any quality control samples and/or reincurred patient samples that were measured alongside a target patient sample.

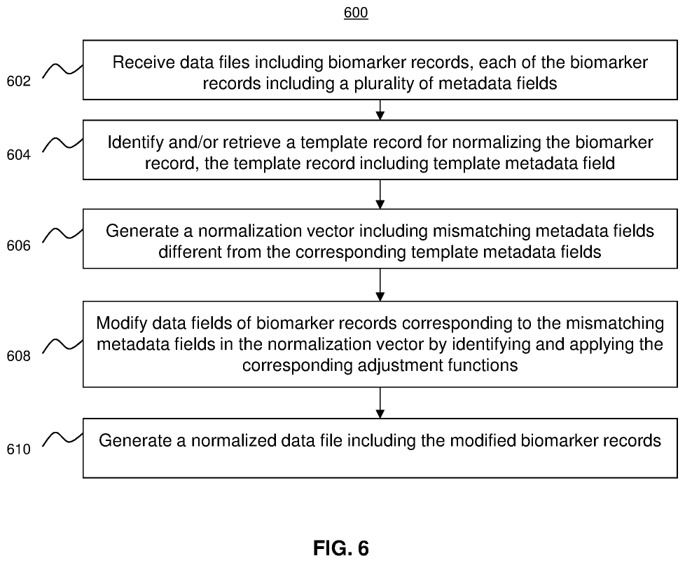

FIG. 6 illustrates a flow chart of a method 600 for normalizing biomarker records, in accordance with disclosed embodiments. Method 600 may be performed at least partially by any one of client devices coupled to one or more servers through a network (e.g., any one of servers 130 and any one of client devices 110, and network 150). For example, in accordance with various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 600 may be performed by a computer having a processor executing commands stored in a memory of the computer (e.g., processors 212 and memories 220). In accordance with various embodiments, the user may activate an application in the client device to access, through the network, a trigger logic engine in the server (e.g., application 222 and trigger logic engine 240). The trigger logic engine may include a modeling tool, a statistics tool, and an imputation tool to retrieve, supply, and process clinical data in real-time, and provide an action recommendation thereof (e.g., modeling tool 242, statistics tool 244, and imputation tool 246).

Further, steps as disclosed in method 600 may include retrieving, editing, and/or storing files in a database that is part of, or is communicably coupled to, the computer, using, inter-alia, a trigger logic engine (e.g., databases 252). Methods consistent with the present disclosure may include at least some, but not all, of the steps illustrated in method 600, performed in a different sequence. Furthermore, methods consistent with the present disclosure may include at least two or more steps as in method 600 performed overlapping in time, or almost simultaneously.

Step 602 may include receiving data files including biomarker records, each of the biomarker records including a plurality of metadata fields. That is, a data file may include a biomarker record that contains a plurality of biomarker metadata fields. Specifically, the biomarker records may be received by streaming module 452 and then sent to data queue 460. For example, in step 602 server 130 may receive biomarker records from a hospital, a clinical laboratory, or a research institute.

Step 604 may include identifying and/or retrieving a template record for normalization, i.e., for normalizing the biomarker records, the template record including template metadata field. This can be accomplished by first extracting the test name from the input biomarker record and then retrieving from template memory 456 the entry with the corresponding test name.

Step 606 may include generating a normalization vector including mismatching biomarker records that have metadata fields different from the template. This logic may be encapsulated or programmed in Metadata Analyzer 454. The normalization vector can be formed by performing an iterative comparison between each metadata field, i.e., between the metadata field of the biomarker record and a corresponding metadata field of the template record, determining if they are equal, and setting the value for that metadata field to ‘1’ if so and to ‘0’ if not. The normalization vector is then of the format: {field1: 1/0, field2: 1/0, . . . , fieldN: 1/0}.

In various embodiments, a biomarker record having biomarker metadata fields may be a mismatching biomarker record (e.g., with respect to the template) when one or more metadata fields of the biomarker record is different from a corresponding or respective metadata field of the template. For example, with reference to FIG. 5, the plurality of metadata fields 520 of the biomarker record 520 may have the refrigeration time field 524 which may be different from a corresponding or respective refrigeration time field of the template. That is, for instance, the refrigeration time field 524 of the biomarker record 520 and the corresponding or respective refrigeration field of the template may not be measured from the same reference time, and as such, the refrigeration time field 524 of the biomarker record 520 and the corresponding or respective refrigeration field of the template may be considered to be mismatched (e.g., further the biomarker record 520 and the template may also be considered as mismatched). In such cases, the value in the normalization vector that corresponds to a refrigeration time field may be set at 0 to indicate that the mismatch.

Step 608 may include modifying data fields of biomarker records in the normalization vector by applying the adjustment functions. That is, an adjustment function may be identified for a mismatching metadata field in the normalization vector, and a data field of the biomarker record corresponding to the mismatching metadata field may be modified by applying the adjustment function to the data field. Specifically, for each metadata field name in the normalization vector, check if the value equals ‘1’, and if it does, then identify and/or retrieve the corresponding adjustment function. For instance, given the biomarker record, extract the test name and then that test name combined with the corresponding metadata field can be used as index into Adjust Functions 458, which may then output the corresponding adjustment function. After the adjustment function is retrieved, it may then be applied to the biomarker record.

Step 610 may include generating a normalized data file including the modified biomarker records after applying all relevant adjustment functions to the input biomarker record.

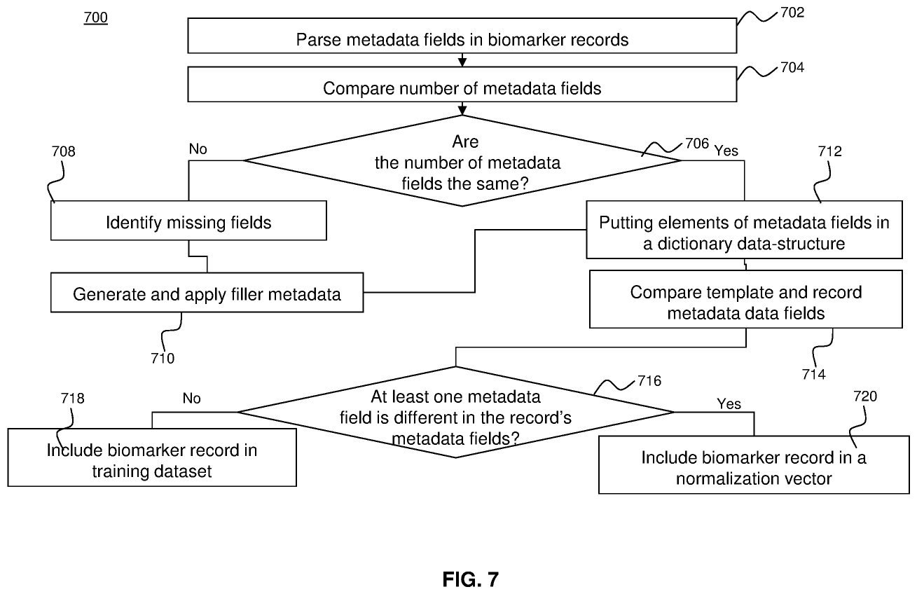

FIG. 7 illustrates a flow chart of a method 700 for identifying unnormalized records based on metadata information, in accordance with disclosed embodiments. Method 700 may be performed at least partially by any one of client devices coupled to one or more servers through a network (e.g., any one of servers 130 and any one of client devices 110, and network 150). For example, in accordance with various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 700 may be performed by a computer having a processor executing commands stored in a memory of the computer (e.g., processors 212 and memories 220). In accordance with various embodiments, the user may activate an application in the client device to access, through the network, a trigger logic engine in the server (e.g., application 222 and trigger logic engine 240). The trigger logic engine may include a modeling tool, a statistics tool, and an imputation tool to retrieve, supply, and process clinical data in real-time, and provide an action recommendation thereof (e.g., modeling tool 242, statistics tool 244, and imputation tool 246).

Further, steps as disclosed in method 700 may include retrieving, editing, and/or storing files in a database that is part of, or is communicably coupled to, the computer, using, inter-alia, a trigger logic engine (e.g., databases 252). Methods consistent with the present disclosure may include at least some, but not all, of the steps illustrated in method 700, performed in a different sequence. Furthermore, methods consistent with the present disclosure may include at least two or more steps as in method 700 performed overlapping in time, or almost simultaneously.

Step 702 may include parsing metadata fields in biomarker records data and step 704 may include comparing the number of metadata fields between the biomarker records data and template data. For example, Metadata Analyzer 454 may read metadata fields in biomarker records and compare number of metadata fields in received biomarker records with samples stored in template memory 456.

Step 706 may include a determination of whether the number of metadata fields are the same. If in step 706 it is determined that the number of metadata fields are not the same (Step 706: No), method 700 may continue to step 708, which may include identifying missing fields, and to step 710, which may include replacing each missing metadata field with a value indicating that it is missing or imputing it with a value derived from records in database 252. For instance, records in database 252 may contain instances where the metadata field of interest is not missing, and hence from these instances, values like the average, median, and mode of the metadata field of interest can be calculated. Such values can then be imputed for the given missing metadata field.

However, if in step 706 it is determined that the number of metadata fields are the same (Step 706: Yes), method 700 may continue to step 712, which may include putting elements of metadata fields in a dictionary data-structure that maps a metadata field to its given value. This could be done for both the template and record data. Step 712 can then continue to step 714, which may include comparing template and record metadata data fields.

Step 716 may include a determination of whether at least one metadata field is different in the record's metadata fields. This can be done by comparing the dictionary data-structure of metadata fields between the template and record data. If in step 716 it is determined that no metadata field is different in the record's metadata fields (Step 716: No), method 700 may continue to step 718, which may include including the biomarker record in a training data set for ML. However, if in step 716 it is determined that at least one metadata field is different in the record's metadata fields (Step 716: Yes), method 700 may continue to step 720, which may include generating a normalization vector for the biomarker record.

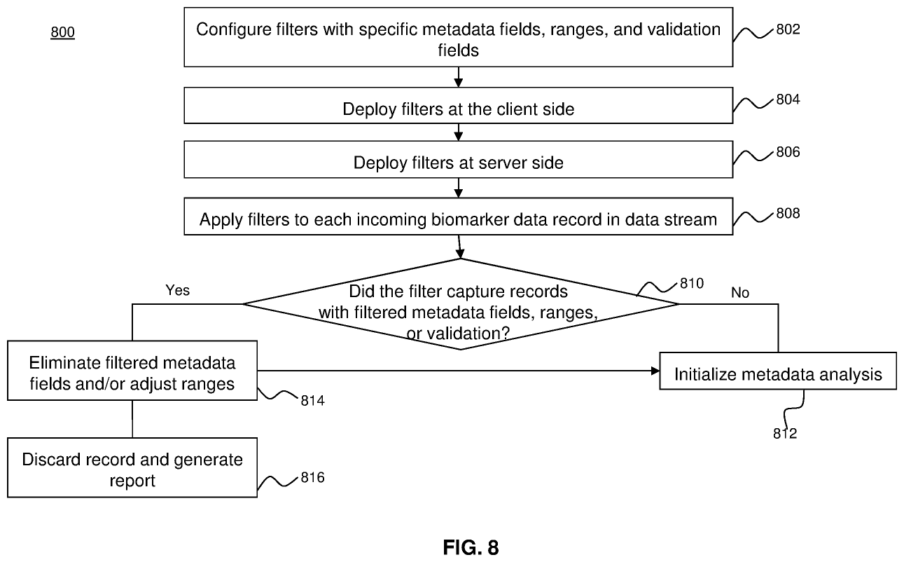

FIG. 8 illustrates a flow chart of a method 800 for filtering records based on metadata information, in accordance with disclosed embodiments. Method 800 may be performed at least partially by any one of client devices coupled to one or more servers through a network (e.g., any one of servers 130 and any one of client devices 110, and network 150). For example, in accordance with various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 800 may be performed by a computer having a processor executing commands stored in a memory of the computer (e.g., processors 212 and memories 220). In accordance with various embodiments, the user may activate an application in the client device to access, through the network, a trigger logic engine in the server (e.g., application 222 and trigger logic engine 240). The trigger logic engine may include a modeling tool, a statistics tool, and an imputation tool to retrieve, supply, and process clinical data in real-time, and provide an action recommendation thereof (e.g., modeling tool 242, statistics tool 244, and imputation tool 246).

Further, steps as disclosed in method 800 may include retrieving, editing, and/or storing files in a database that is part of, or is communicably coupled to, the computer, using, inter-alia, a trigger logic engine (e.g., databases 252). Methods consistent with the present disclosure may include at least some, but not all, of the steps illustrated in method 800, performed in a different sequence. Furthermore, methods consistent with the present disclosure may include at least two or more steps as in method 800 performed overlapping in time, or almost simultaneously.

Step 802 may include configuring filters with specific metadata fields, ranges, and validation values. For instance, certain metadata fields may be categorical and can only be equal to a value from a prespecified set of entries. In some instances, other metadata fields may be continuous, and their values must exist within a prespecified range.

Step 804 may include deploying filters at the client side and step 806 may include deploying filters at server side. These filters may be implemented as software modules and may be the first pieces of logic to be applied on the input in the normalization pipeline. Step 808 may include applying filters to each incoming biomarker data record in the data stream. For example, filters deployed in steps 804 and/or 806 may be applied to incoming biomarker data in step 808.

Step 810 may include a determination of whether the filter captured records with filtered metadata fields, ranges, or validation. If it is determined that the filter did capture records (Step 810: Yes), method 800 may continue to step 814, which may include eliminating filtered metadata fields and/or adjust range, and step 816, which may include discarding records and generating error log messages that stored in a database. However, if it is determined that the filter did not capture records (Step 810: No), method 800 may continue to step 812, which may include initializing metadata analysis.

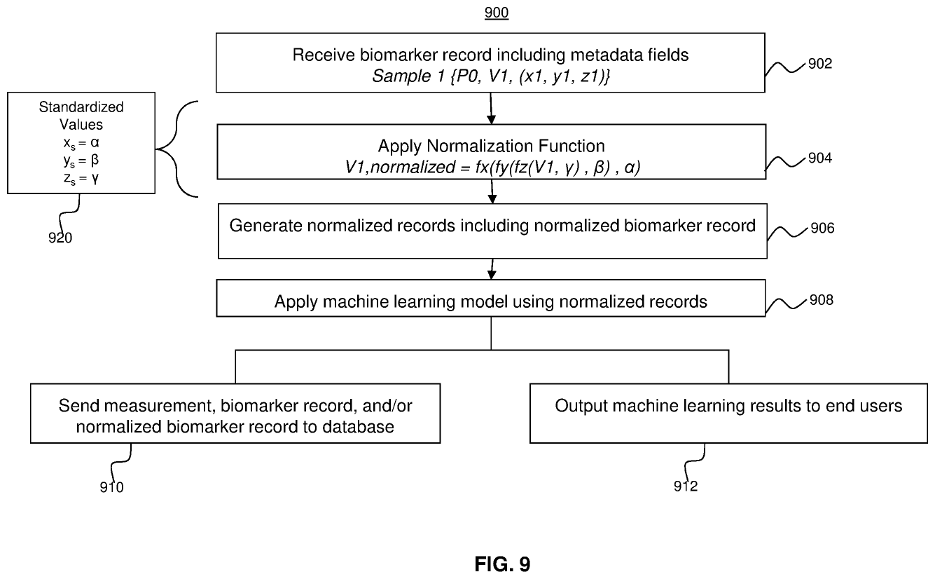

FIG. 9 illustrates a flow chart of a method 900 for deployment of normalization, in accordance with disclosed embodiments. Method 900 may be performed at least partially by any one of client devices coupled to one or more servers through a network (e.g., any one of servers 130 and any one of client devices 110, and network 150). For example, in accordance with various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 900 may be performed by a computer having a processor executing commands stored in a memory of the computer (e.g., processors 212 and memories 220). In accordance with various embodiments, the user may activate an application in the client device to access, through the network, a trigger logic engine in the server (e.g., application 222 and trigger logic engine 240). The trigger logic engine may include a modeling tool, a statistics tool, and an imputation tool to retrieve, supply, and process clinical data in real-time, and provide an action recommendation thereof (e.g., modeling tool 242, statistics tool 244, and imputation tool 246).

Further, steps as disclosed in method 900 may include retrieving, editing, and/or storing files in a database that is part of, or is communicably coupled to, the computer, using, inter-alia, a trigger logic engine (e.g., databases 252). Methods consistent with the present disclosure may include at least some, but not all, of the steps illustrated in method 900, performed in a different sequence. Furthermore, methods consistent with the present disclosure may include at least two or more steps as in method 900 performed overlapping in time, or almost simultaneously.

Step 902 may include receiving a biomarker record including metadata fields. For example, a biomarker record of “Sample 1{P₀, V₁, (x₁, y₁, z₁)}” may be received in step 902. P₀ refers to the name of a specific parameter (e.g IL-6), V₁ corresponds to that parameter's value, and (x₁, y₁, z₁) refers to the value of 3 hypothetical metadata fields labeled x, y, and z.

Step 904 may include applying a composition of normalization functions, such as “V_{1,normalized}=fₓ(f_{y}(f_{z}(V₁, γ), β), α),” where the functions may be parameterized based on standardized values 920. In this step, f refers to the adjustment function for metadata field x,f_{y} refers to the adjustment function for metadata field y, and f_{z} refers to the adjustment function for metadata field z. V_{1,normalized} is specifically calculated as follows: f_{z} is first applied to V₁ with respect to γ; the output of this is then fed into f_{y} and applied with respect to β; and finally the output of this is fed into fₓ and applied with respect to α.

Step 906 may include generating normalized records including normalized biomarker records. For instance, the normalized values generated for input parameters can be assembled into a single row normalized record. Alternatively, or additionally, step 906 may include modifying data fields of biomarker records by identifying and applying the corresponding adjustment functions.

Step 908 may include applying an existing ML model to normalized records. Step 908 may also include training a new ML model exclusively using normalized records.

Step 910 may include sending measurement, biomarker record, and/or normalized biomarker record to database. For example, server 130 may send normalized biomarker records to database 252. Step 912 may include outputting machine learning results to end users. For instance, server 130 may output machine learning results to client devices 110.

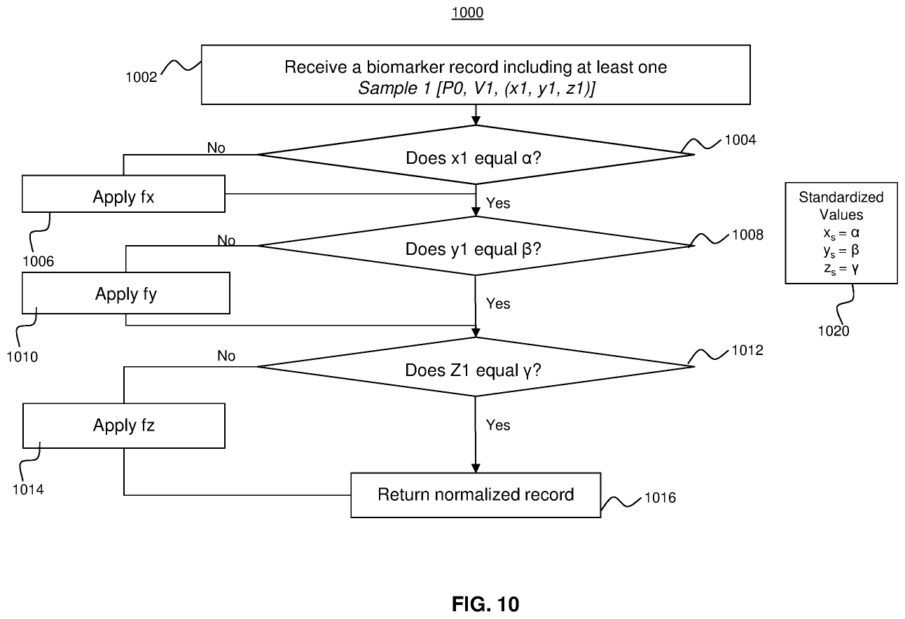

FIG. 10 illustrates a flow chart of a process for applying normalization functions, in accordance with disclosed embodiments. Method 1000 may be performed at least partially by any one of client devices coupled to one or more servers through a network (e.g., any one of servers 130 and any one of client devices 110, and network 150). For example, in accordance with various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 1000 may be performed by a computer having a processor executing commands stored in a memory of the computer (e.g., processors 212 and memories 220). In accordance with various embodiments, the user may activate an application in the client device to access, through the network, a trigger logic engine in the server (e.g., application 222 and trigger logic engine 240). The trigger logic engine may include a modeling tool, a statistics tool, and an imputation tool to retrieve, supply, and process clinical data in real-time, and provide an action recommendation thereof (e.g., modeling tool 242, statistics tool 244, and imputation tool 246).

Further, steps as disclosed in method 1000 may include retrieving, editing, and/or storing files in a database that is part of, or is communicably coupled to, the computer, using, inter-alia, a trigger logic engine (e.g., databases 252). Methods consistent with the present disclosure may include at least some, but not all, of the steps illustrated in method 1000, performed in a different sequence. Furthermore, methods consistent with the present disclosure may include at least two or more steps as in method 1000 performed overlapping in time, or almost simultaneously.

Step 1002 Receiving a biomarker record including at least one Sample 1 with parameters [P₀, V₁, (x₁, y₁, z₁)]. As previously discussed with respect to FIG. 9, P₀ refers to the name of a specific parameter (e.g IL-6), V₁ corresponds to that parameter's value, and (x₁, y₁, z₁) refers to the value of 3 hypothetical metadata fields labeled x, y, and z.

Step 1004 may include a determination of whether metadata field x₁ matches a standardized value α, which may be stored in standardized values 1020. If it is determined that the metadata field x₁ does not match value α (step 1004: No), method 1000 may continue to step 1006, which may include applying a first adjustment function fₓ. Function fₓ may include one or more normalization functions for data manipulation. For instance, function fₓ may include manipulations on floating point numbers that are represented internally using a binary radix. Alternatively, or additionally, function f_{x,may} transform data based on a z-score or t-score for standardization. In various embodiments, function fₓ may rescale data to have values between 0 and 1. However, if it is determined that metadata field x₁ matches value α (step 1004: Yes), method 1000 may continue to step 1008.

Step 1008 may include a determination of whether metadata field y₁ matches a standardized value β, which may be stored in standardized values 1020. If it is determined that the metadata field y₁ does not match value β (step 1008: No), method 1000 may continue to step 1010, which may include applying a first adjustment function f_{y}. Function f_{y} may include normalization functions, data transformations, or rescaling functions, like function fₓ. However, if it is determined that metadata field y₁ matches value α (step 1008: Yes), method 1000 may continue to step 1012.

Step 1012 may include a determination of whether metadata field z₁ matches a standardized value γ, which may be stored in standardized values 1020. If it is determined that the metadata field z₁ does not match value γ (step 1012: No), method 1000 may continue to step 1014, which may include applying a first adjustment function f_{z}. Function f_{z} may include normalization functions, data transformations, or rescaling functions, like function fₓ. However, if it is determined that metadata field z₁ matches value γ (step 1012: Yes), method 1000 may continue to step 1016, which may include returning the normalized record.

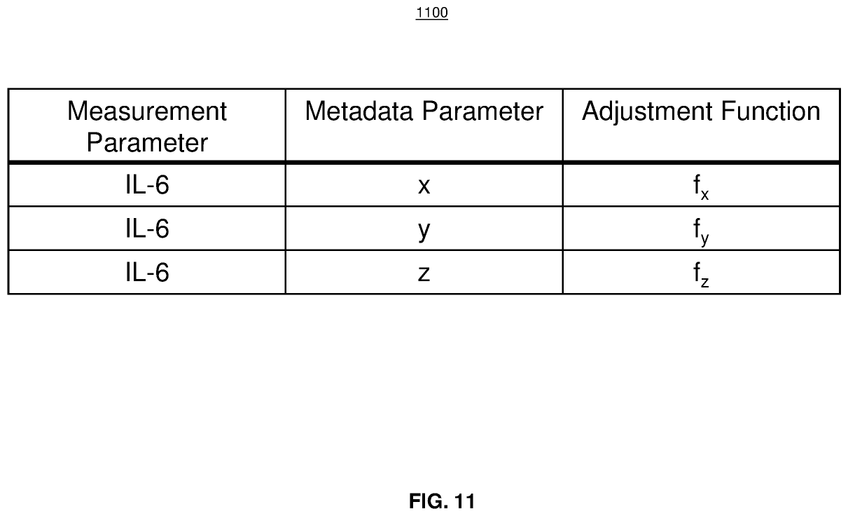

FIG. 11 illustrates an exemplary adjustment function table 1100, in accordance with disclosed embodiments. Function table 1100 associates a measurement parameter, such as a biomarker, with a metadata parameter and an adjustment function.

An adjustment function for a metadata parameter such as sample refrigeration time at 4 degrees Celsius for a biomarker such as IL-6 may be constructed as follows: for a set of subjects, draw a blood sample in a Lithium Heparin Plasma Separator Tube (PST), process it, and store a portion of the plasma in a −80 degree Celsius freezer. After that, store the PST tube for each subject in a 4 degrees Celsius refrigerator. For each subsequent day until 1 week passes, extract a portion of the plasma from each PST tube and store it in a −80 degree Celsius freezer. At the end of the week, take each sample for each patient that was frozen at days 1, 2, 3, . . . , 7 and measure it's IL-6 concentration, where each sample is measured on the same plate and lot ID. Once this is completed, one may construct a linear mixed effects model of the form y=Xβ+Zu+ε, where IL-6 concentration is treated as the dependent variable, and refrigeration time is modeled as a fixed effect and each patient is modeled as a random effect. For a given concentration c of IL-6 that was refrigerated for time t, the corresponding adjustment function would then be f(c, t)=c+βt.

An adjustment function for a metadata parameter such lot ID for a biomarker such as IL-6 may be constructed as follows: If we have n lots (i.e., lot 1, lot 2, . . . , lot n), measure the concentration of m quality control samples of fixed concentration spanning the dynamic range of the assay for lot i and lot i+1 for all i in {1 . . . n}. Specifically, on a given plate, m quality control samples would be run in duplicate, one set using lot i's components and one set using lot i+1's components. A linear or polynomial function of degree k can then be fit between the concentration of each quality control sample from lot i and lot i+1. For instance, this procedure could yield a function f_{i,i+1}(cᵢ₊₁)=βcᵢ₊₁ that specifies how to transform a concentration sourced from lot i+1 to a concentration effectively sourced from lot i. To determine the adjustment function between an arbitrary lot i and lot j, one may compose the functions between each sequential group of lots as follows: f_{i,i+1}(f_{i+1,i+2}( . . . f_{j-1,j}(Cⱼ))).

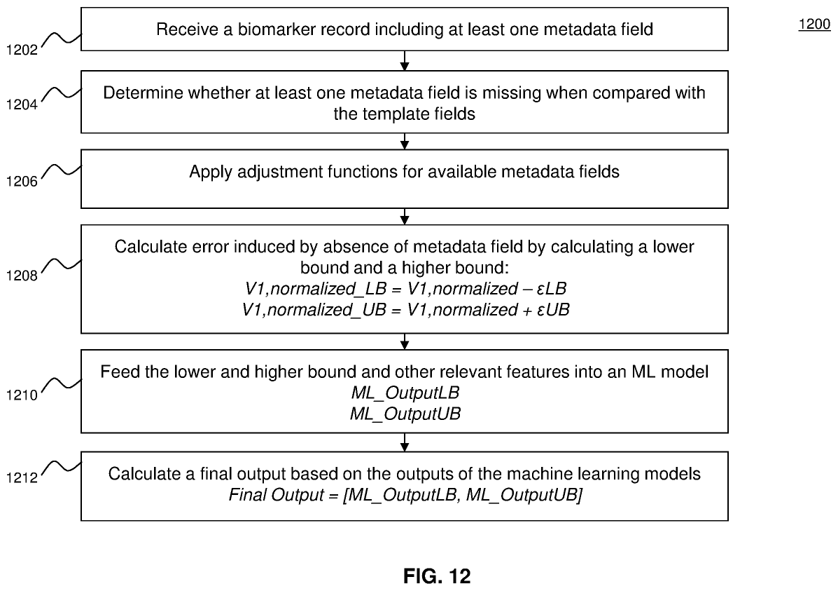

FIG. 12 illustrates a flow chart of a method 1200 for calculating errors based on missing metadata fields, in accordance with disclosed embodiments. Method 1200 may be performed at least partially by any one of client devices coupled to one or more servers through a network (e.g., any one of servers 130 and any one of client devices 110, and network 150). For example, in accordance with various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 1200 may be performed by a computer having a processor executing commands stored in a memory of the computer (e.g., processors 212 and memories 220). In accordance with various embodiments, the user may activate an application in the client device to access, through the network, a trigger logic engine in the server (e.g., application 222 and trigger logic engine 240). The trigger logic engine may include a modeling tool, a statistics tool, and an imputation tool to retrieve, supply, and process clinical data in real-time, and provide an action recommendation thereof (e.g., modeling tool 242, statistics tool 244, and imputation tool 246).

Further, steps as disclosed in method 1200 may include retrieving, editing, and/or storing files in a database that is part of, or is communicably coupled to, the computer, using, inter-alia, a trigger logic engine (e.g., databases 252). Methods consistent with the present disclosure may include at least some, but not all, of the steps illustrated in method 1200, performed in a different sequence. Furthermore, methods consistent with the present disclosure may include at least two or more steps as in method 1200 performed overlapping in time, or almost simultaneously.

Step 1202 may include receiving a biomarker record including at least one metadata field. For example, step 1202 may include receiving data files including biomarker records, each of the biomarker records including a plurality of metadata fields.

Step 1204 may include determining whether at least one metadata field is missing when compared with the template fields. For example, step 1204 may include performing method 700 to determine the number of metadata fields in received and template records to determine if they are the same.

Step 1206 may include applying the corresponding adjustment functions for available metadata fields. For example, step 1206 may include performing method 1000 to apply normalization functions when finding un-matching metadata fields.

Step 1208 may include calculating an error induced by absence of metadata field by calculating a lower bound (V_{1,normalized_LB}=V_{1,normalized}−ε_{LB}) and a higher bound (V_{1,normalized_UB}=V_{I,normalized}+ε_{UB}). The error induced by the absence of a specific metadata field can be calculated by assembling all normalized records in database 252 where that specific metadata field is present; then, setting the metadata field to missing and recalculating the normalized values of the records under this artificial constraint; then comparing these artificially calculated normalized values to the true normalized values in the case where the metadata field is not missing; the difference between these two normalized values then provides an error distribution that can be used to define ε_{LB} and ε_{UB}.

Step 1210 may include separately feeding V_{1,normalized_LB} and V_{1,normalized_UB} and other relevant features into an ML model which would output two values: ML_Output_{LB} and ML_Output_{UB}. These values provide an estimate of the error induced by the missing metadata fields on the ML model.

Step 1212 may include calculating a final output based on the outputs of the machine learning models. For example, a final output may be defined by Final Output=[ML_Output_{LB}, ML_Output_{UB}]

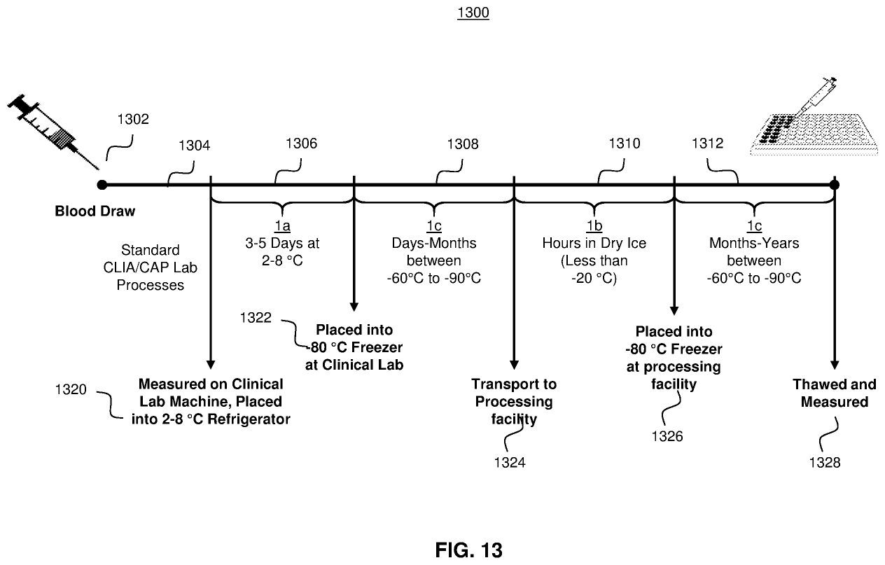

FIG. 13 illustrates a timeline 1300 of blood draw records, in accordance with disclosed embodiments. Timeline 1300 may include with a blood draw time 1302, which may be followed by a standard lab interval 1304. Then after a sample is measured in time 1320, an interval 1306 may follow. Then, a sample may be placed into a freezer in time 1322. After time 1322, an interval 1308 may follow with storage. Later, at a time 1324 a sample may be transported to a processing facility. At the processing facility, the sample may enter an interval 1310 until a time 1326, in which the sample may be placed into a freezer in a processing facility.

After time 1326, the sample may enter an interval 1312 of storage that may be culminated in a time 1328, in which the sample is thawed and measured.

Timeline 1300 illustrates how one single biomarker measurement may include multiple dynamic variables that describe its collection and storage. These variables may be relevant for certain ML algorithms to create high quality datasets for training and/or validation. The disclosed embodiments improve technical fields of data processing by providing systems and methods to effectively normalize biomarker records so they can be used in ML training or validation.

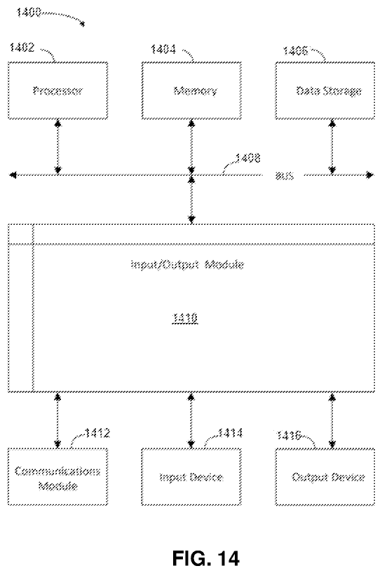

FIG. 14 is a block diagram illustrating an exemplary computer system 1400 with which the client device 110 and server 130 of FIGS. 1 and 2, and the methods described in FIGS. 5-13 can be implemented. In certain aspects, the computer system 1400 may be implemented using hardware or a combination of software and hardware, either in a dedicated server, or integrated into another entity, or distributed across multiple entities.

Computer system 1400 (e.g., client device 110 and server 130) includes a bus 1408 or other communication mechanism for communicating information, and a processor 1402 (e.g., processors 212) coupled with bus 1408 for processing information. By way of example, the computer system 1400 may be implemented with one or more processors 1402. Processor 1402 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1400 can include, in addition to hardware, code that creates an execution environment for the computer program in question, e.g., code that constitutes processor firmware, a protocol stack, a database management system, an operating system, or a combination of one or more of them stored in an included memory 1404 (e.g., memories 220), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to bus 1408 for storing information and instructions to be executed by processor 1402. The processor 1402 and the memory 1404 can be supplemented by, or incorporated in, special purpose logic circuitry.

The instructions may be stored in the memory 1404 and implemented in one or more computer program products, i.e., one or more modules of computer program instructions encoded on a computer-readable medium for execution by, or to control the operation of, the computer system 1400, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multi paradigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 1404 may also be used for storing temporary variables or other intermediate information during execution of instructions to be executed by processor 1402.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 1400 further includes a data storage device 1406 such as a magnetic disk or optical disk, coupled to bus 1408 for storing information and instructions. Computer system 1400 may be coupled via input/output module 1410 to various devices. Input/output module 1410 can be any input/output module. Exemplary input/output modules 1410 include data ports such as USB ports. The input/output module 1410 is configured to connect to a communications module 1412. Exemplary communications modules 1412 (e.g., communications modules 218) include networking interface cards, such as Ethernet cards and modems. In certain aspects, input/output module 1410 is configured to connect to a plurality of devices, such as an input device 1414 (e.g., input device 214) and/or an output device 1416 (e.g., output device 216). Exemplary input devices 1414 include a keyboard and a pointing device, e.g., a mouse or a trackball, by which a user can provide input to the computer system 1400. Other kinds of input devices 1414 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 1416 include display devices, such as an LCD (liquid crystal display) monitor, for displaying information to the user.

According to one aspect of the present disclosure, the client device 110 and server 130 can be implemented using a computer system 1400 in response to processor 1402 executing one or more sequences of one or more instructions contained in memory 1404. Such instructions may be read into memory 1404 from another machine-readable medium, such as data storage device 1406. Execution of the sequences of instructions contained in main memory 1404 causes processor 1402 to perform the process steps described herein. One or more processors in a multi-processing arrangement may also be employed to execute the sequences of instructions contained in memory 1404. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1400 can include clients and servers. A client and server are generally remote from each other and typically interact through a communication network. The relationship of client and server arises by virtue of computer programs running on the respective computers and having a client-server relationship to each other. Computer system 1400 can be, for example, and without limitation, a desktop computer, laptop computer, or tablet computer. Computer system 1400 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 1402 for execution. Such a medium may take many forms, including, but not limited to, non-volatile media, volatile media, and transmission media. Non-volatile media include, for example, optical or magnetic disks, such as data storage device 1406. Volatile media include dynamic memory, such as memory 1404. Transmission media include coaxial cables, copper wire, and fiber optics, including the wires that include bus 1408. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

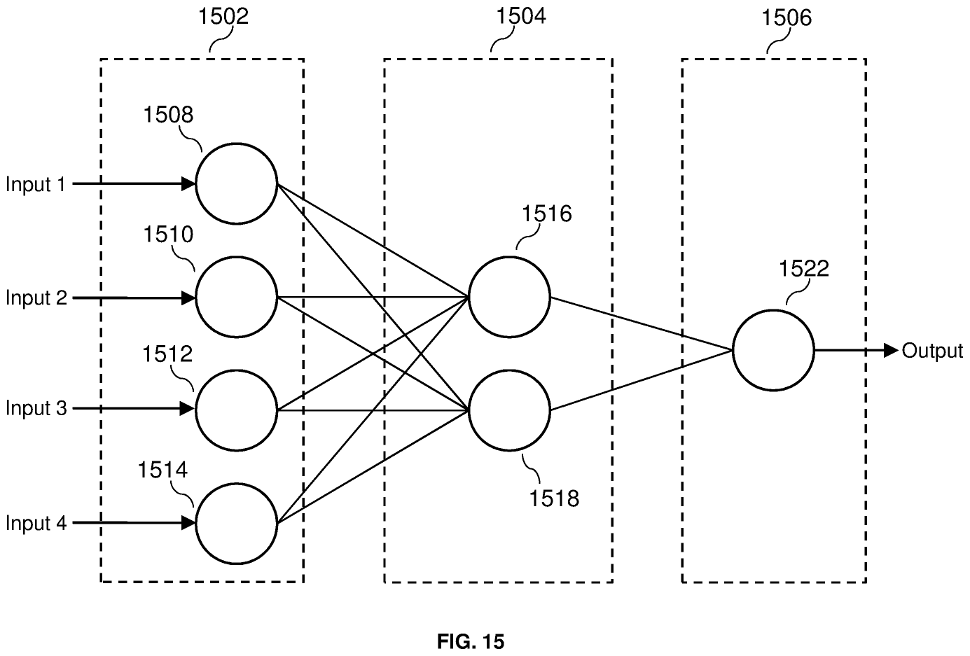

As discussed above, the trigger logic engine (e.g., the trigger logic engine 240 in FIG. 2, the trigger system in FIG. 3, etc.) may include machine learning (ML) models, artificial intelligence (AI) models, neural network (NN) models, any combination thereof, and/or the like. For example, the logic engine may be implemented using a neural network such as but not limited to a deep neural network (DNN), a convolutional neural network (CNN), etc. That is, the machine learning of the trigger logic engine may be performed at least in part using a AI model, NN model, ML model, or combination thereof. FIG. 15 illustrates an example neural network that can be used to implement the logic engine according to various embodiments of the present disclosure. It is to be understood that FIG. 15 is a non-limiting example illustration and that other types of neural networks or AI/ML algorithms and models can be used to implement the logic engines according to the various embodiments of the present disclosure.

As shown, the artificial neural network 1500 includes three layers—an input layer 1502, a hidden layer 1504, and an output layer 1506. Each of the layers 1502, 1504, and 1506 may include one or more nodes. For example, the input layer 1502 includes nodes 1508-1514, the hidden layer 1504 includes nodes 1516-1518, and the output layer 1506 includes a node 1522. In this example, each node in a layer is connected to every node in an adjacent layer. For example, the node 1508 in the input layer 1502 is connected to both of the nodes 1516, 1518 in the hidden layer 1504. Similarly, the node 1516 in the hidden layer is connected to all of the nodes 1508-1514 in the input layer 1502 and the node 1522 in the output layer 1506. Although only one hidden layer is shown for the neural network 1500, it has been contemplated that the neural network 1500 used to implement the logic engine disclosed herein may include as many hidden layers as necessary or desired.

In this example, the neural network 1500 receives a set of input values and produces an output value. Each node in the input layer 1502 may correspond to a distinct input value. For example, when the neural network 1500 is used to implement the logic engine disclosed herein, each node in the input layer 1502 may correspond to the input data {Xi(tx)}.

In various embodiments, each of the nodes 1516-1518 in the hidden layer 1504 generates a representation, which may include a mathematical computation (or algorithm) that produces a value based on the input values received from the nodes 1508-1514. The mathematical computation may include assigning different weights to each of the data values received from the nodes 1508-1514. The nodes 1516 and 1518 may include different algorithms and/or different weights assigned to the data variables from the nodes 1508-1514 such that each of the nodes 1516-1518 may produce a different value based on the same input values received from the nodes 1508-1514. In various embodiments, the weights that are initially assigned to the features (or input values) for each of the nodes 1516-1518 may be randomly generated (e.g., using a computer randomizer). The values generated by the nodes 1516 and 1518 may be used by the node 1522 in the output layer 1506 to produce an output value for the neural network 1500. When the neural network 1500 is used to implement the logic engine disclosed herein, the output value produced by the neural network 1500 may indicate the imputed data {Yi(tx)}.

The neural network 1500 may be trained by using training data. For example, the training data herein may be training dataset from the training database 252-2. By providing training data to the neural network 1500, the nodes 1516-1518 in the hidden layer 1504 may be trained (adjusted) such that an optimal output is produced in the output layer 1506 based on the training data. By continuously providing different sets of training data, and penalizing the neural network 1500 when the output of the neural network 1500 is incorrect, the neural network 1500 (and specifically, the representations of the nodes in the hidden layer 1504) may be trained (adjusted) to improve its performance in data normalization. Adjusting the neural network 1500 may include adjusting the weights associated with each node in the hidden layer 1504.

Although the above discussions pertain to a neural network as an example of logic engine, it is understood that other types of AI/ML methods may also be suitable to implement the various aspects of the present disclosure. For example, support vector machines (SVMs) may be used to implement machine learning. SVMs are a set of related supervised learning methods used for classification and regression. A SVM training algorithm-which may be a non-probabilistic binary linear classifier—may build a model that predicts whether a new example falls into one category or another. As another example, Bayesian networks may be used to implement machine learning. A Bayesian network is an acyclic probabilistic graphical model that represents a set of random variables and their conditional independence with a directed acyclic graph (DAG). The Bayesian network could present the probabilistic relationship between one variable and another variable. Another example is a machine learning engine that employs a decision tree learning model to conduct the machine learning process. In some instances, decision tree learning models may include classification tree models, as well as regression tree models. In various embodiments, the machine learning engine employs a Gradient Boosting Machine (GBM) model (e.g., XGBoost) as a regression tree model. Other machine learning techniques may be used to implement the machine learning engine, for example via Random Forest or Deep Neural Networks. Other types of machine learning algorithms are not discussed in detail herein for reasons of simplicity and it is understood that the present disclosure is not limited to a particular type of machine learning.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C. To the extent that the term “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products. Other variations are within the scope of the following claims.

### Recitations of Various Embodiments of the Present Disclosure

Embodiment 1: A computer implemented method for biomarker data normalization in training data sets, the method comprising: receiving a data file comprising a biomarker record that includes a plurality of biomarker metadata fields; identifying a template record for normalizing the biomarker record, the template record comprising template metadata fields; generating a normalization vector comprising a mismatching metadata field that mismatches a corresponding template metadata field in the template record; identifying an adjustment function for the mismatching metadata fields; modifying a data field of the biomarker record by applying the adjustment function to the data field, the data field corresponding to the mismatching metadata field in the normalization vector; and generating a normalized data file comprising the modified biomarker record.

Embodiment 2: The method of embodiment 1, wherein the plurality of biomarker metadata fields comprise a type of specimen collected field, the type of specimen collected field indicating at least one of blood, urine, or cerebrospinal fluid.

Embodiment 3: The method of embodiment 1 or 2, wherein the plurality of biomarker metadata fields comprise: a source of the measurement field, the source measurement field comprising at least one of vein or artery; and a type of tube field.

Embodiment 4: The method of any of embodiments 1-3, wherein the plurality of biomarker metadata fields comprise a first time lapse field, the time lapse field comprising a time between sample collection time and measurement time.

Embodiment 5: The method of embodiment 4, wherein the plurality of biomarker metadata fields comprise a second time lapse field, the second time lapse field comprising a time between sample measurement time and sample refrigeration.

Embodiment 6: The method of embodiment 5, wherein the plurality of biomarker metadata fields comprise: a third time lapse field, the third time lapse field comprising a time between sample offsite refrigeration and sample offsite freezer placement; and a fourth time lapse, the fourth time lapse comprising a time between sample onsite freezer time and sample onsite measurement time.

Embodiment 7: The method of any of embodiments 1-6, wherein the plurality of biomarker metadata fields comprise: a machine identifier field; and a measurement process field.

Embodiment 8: The method of any of embodiments 1-7, wherein the plurality of biomarker metadata fields comprise: an offsite refrigeration temperature field, the offsite refrigeration temperature field comprising a plurality of temperature values experienced by samples while stored in an offsite refrigerator; and an offsite freezer temperature field, the offsite freezer temperature field comprising a plurality of temperature values experienced by samples while stored in offsite freezers.

Embodiment 9: The method of any of embodiments 1-8, wherein the plurality of biomarker metadata fields comprise a temperature during transport field, the temperature during transport field comprising temperature values samples experience while being transported from the offsite freezer to onsite freezer.

Embodiment 10: The method of any of embodiments 1-9, wherein the plurality of biomarker metadata field comprises a measurement process field.

Embodiment 11: The method of any of embodiments 1-10, wherein the plurality of biomarker metadata fields comprise a number of freeze-thaw cycles field and an equipment of collection field.

Embodiment 12: The method of any of embodiments 1-11, wherein generating the normalization vector comprises labeling the biomarker record based on the mismatching metadata fields.

Embodiment 13: The method of any of embodiments 1-12, wherein the plurality of biomarker metadata fields comprises at least one of a unique IDs field that is sourced from a specific analysis used to measure a parameter, a quality control sample field, or a reincurred patient field that comprises values measured alongside a target patient sample.

Embodiment 14: The method of any of embodiments 1-13, wherein the operations further comprise: determining whether the plurality of biomarker metadata fields fail to comprise each one of the template metadata fields; and in response to determining the plurality of biomarker metadata fields fail to comprise each one of the template metadata fields, calculating an associated error for a measurement associated with the biomarker record.

Embodiment 15: The method of embodiment 14, wherein the operations further comprise incorporating cumulative errors for the measurement induced by missing biomarker metadata fields into an input of a machine learning model.

Embodiment 16: The method of embodiment 15, wherein the operations further comprise calculating an uncertainty interval for a machine learning prediction generated from measurements with at least one missing biomarker metadata field.

Embodiment 17: The method of any of embodiments 1-16, wherein the operations further comprise: building a machine learning model based on the normalized data file; and inputting the modified data field into a static machine learning model and feeding an output of the static machine learning model to an end user.

Embodiment 18: The method of embodiment 17, wherein the static machine learning model predicts dysregulated host response.

Embodiment 19: A system, comprising: one or more memory devices; and one or more processors coupled to the one or more memory devices storing instructions that configure the one or more processors to perform the methods of embodiments 1-18.

Embodiment 20: A non-transitory computer-readable medium (CRM) storing instructions that when executed by one or more processors, cause the one or more processors to perform the methods of embodiments 1-18.

What is claimed is:

1. A system for biomarker data normalization in training datasets, the system comprising:
one or more processors; and
one or more memory devices storing instructions that configure the one or more processors to perform operations comprising:
receiving a data file comprising a biomarker record that includes a plurality of biomarker metadata fields;
identifying a template record for normalizing the biomarker record, the template record comprising template metadata fields;
generating a normalization vector comprising a mismatching metadata field that mismatches a corresponding template metadata field in the template record;
identifying an adjustment function for the mismatching metadata fields;
modifying a data field of the biomarker record by applying the adjustment function to the data field, the data field corresponding to the mismatching metadata field in the normalization vector; and
generating a normalized data file comprising the modified biomarker record.

2. The system of claim 1, wherein the plurality of biomarker metadata fields comprise a type of specimen collected field, the type of specimen collected field indicating at least one of blood, urine, or cerebrospinal fluid.

3. The system of claim 1, wherein the plurality of biomarker metadata fields comprise:
a source of the measurement field, the source measurement field comprising at least one of vein or artery; and
a type of tube field.

4. The system of claim 1, wherein the plurality of biomarker metadata fields comprise a first time lapse field, the time lapse field comprising a time between sample collection time and measurement time.

5. The system of claim 4, wherein the plurality of biomarker metadata fields comprise a second time lapse field, the second time lapse field comprising a time between sample measurement time and sample refrigeration.

6. The system of claim 5, wherein the plurality of biomarker metadata fields comprise:
a third time lapse field, the third time lapse field comprising a time between sample offsite refrigeration and sample offsite freezer placement; and
a fourth time lapse, the fourth time lapse comprising a time between sample onsite freezer time and sample onsite measurement time.

7. The system of claim 1, wherein the plurality of biomarker metadata fields comprise:
a machine identifier field; and
a measurement process field.

8. The system of claim 1, wherein the plurality of biomarker metadata fields comprise:
an offsite refrigeration temperature field, the offsite refrigeration temperature field comprising a plurality of temperature values experienced by samples while stored in an offsite refrigerator; and
an offsite freezer temperature field, the offsite freezer temperature field comprising a plurality of temperature values experienced by samples while stored in offsite freezers.

9. The system of claim 1, wherein the plurality of biomarker metadata fields comprise a temperature during transport field, the temperature during transport field comprising temperature values samples experience while being transported from the offsite freezer to onsite freezer.

10. The system of claim 1, wherein the plurality of biomarker metadata field comprise a measurement process field.

11. The system of claim 1, wherein the plurality of biomarker metadata fields comprise a number of freeze-thaw cycles field and an equipment of collection field.

12. The system of claim 1, wherein generating the normalization vector comprises labeling the biomarker record based on the mismatching metadata fields.

13. The system of claim 1, wherein the plurality of biomarker metadata fields comprises at least one of a unique IDs field that is sourced from a specific analysis used to measure a parameter, a quality control sample field, or a reincurred patient field that comprises values measured alongside a target patient sample.

14. The system of claim 1, wherein the operations further comprise:
determining whether the plurality of biomarker metadata fields fail to comprise each one of the template metadata fields; and
in response to determining the plurality of biomarker metadata fields fail to comprise each one of the template metadata fields, calculating an associated error for a measurement associated with the biomarker record.

15. The system of claim 14, wherein the operations further comprise incorporating cumulative errors for the measurement induced by missing biomarker metadata fields into an input of a machine learning model.

16. The system of claim 15, wherein the operations further comprise calculating an uncertainty interval for a machine learning prediction generated from measurements with at least one missing biomarker metadata field.

17. The system of claim 1, wherein the operations further comprise:
building a machine learning model based on the normalized data file; and
inputting the modified data field into a static machine learning model and feeding an output of the static machine learning model to an end user.

18. The system of claim 17, wherein the static machine learning model predicts dysregulated host response.

19. A computer implemented method for biomarker data normalization in training data sets, the method comprising:
receiving a data file comprising a biomarker record that includes a plurality of biomarker metadata fields;
identifying a template record for normalizing the biomarker record, the template record comprising template metadata fields;
generating a normalization vector comprising a mismatching metadata field that mismatches a corresponding template metadata field in the template record;
identifying an adjustment function for the mismatching metadata fields;
modifying a data field of the biomarker record by applying the adjustment function to the data field, the data field corresponding to the mismatching metadata field in the normalization vector; and
generating a normalized data file comprising the modified biomarker record.

20. A non-transitory computer-readable medium storing instructions that when executed by one or more processors, cause the one or more processors to perform operations comprising:
receiving a data file comprising a biomarker record that includes a plurality of biomarker metadata fields;
identifying a template record for normalizing the biomarker record, the template record comprising template metadata fields;
generating a normalization vector comprising a mismatching metadata field that mismatches a corresponding template metadata field in the template record;
identifying an adjustment function for the mismatching metadata fields;
modifying a data field of the biomarker record by applying the adjustment function to the data field, the data field corresponding to the mismatching metadata field in the normalization vector; and
generating a normalized data file comprising the modified biomarker record.
